(19) United States
(12) Patent Application Publication
(10) Pub. No.: US 2024/0216217 A1
(43) Pub. Date: Jul. 4, 2024

(54) MULTIFUNCTIONAL CARDIOPULMONARY RESUSCITATION MACHINERY EQUIPMENT AND OPERATION METHOD THEREOF

(71) Applicant: Wenzhou Safety (Emergency) Institute of Tianjin University, Zhejiang Province (CN)

(72) Inventors: Bin Fan, Tianjin (CN); Huiquan Wang, Tianjin (CN); Shuai Ma, Shandong (CN); Haojun Fan, Beijing (CN); Shike Hou, Beijing (CN); Wenlong Dong, Tianjin (CN)

(21) Appl. No.: 18/536,009

(22) Filed: Dec. 11, 2023

(30) Foreign Application Priority Data
Dec. 31, 2022 (CN) 2022117338452

(51) Int. Cl. A61H 31/00; A61F 17/00

(52) U.S. Cl. CPC A61H 31/004; A61F 17/00; A61H 31/008; A61H 2201/107; A61N 1/39044

(57) ABSTRACT

A multifunctional cardiopulmonary resuscitation machinery equipment may include a first-aid kit body, and a pressing device, a ventilation device, a monitoring device, a defibrillation device and a control device which are installed on the first-aid kit body; the upper end surface of the first-aid kit body is concavely provided with a curved pillow opening; a belt is counter-pulled between the edges of both sides of the inner side surface of the protecting pad; the pressing device and the defibrillation device are integrated and sleeved on the belt; the ventilation device comprises an oxygen generation pump, an oxygen mask and a gas transmission pipe which are integrated inside the first-aid kit body, and an output port of the oxygen generation pump penetrates through the first-aid kit body and is connected with the oxygen mask.

### TECHNICAL FIELD

The present application relates to the field of cardiopulmonary resuscitation rescue equipment, in particular to a multifunctional cardiopulmonary resuscitation machinery equipment and an operation method thereof.

### BACKGROUND

Cardiopulmonary resuscitation, abbreviated as CPR, is a life-saving technique for arrested heart and breathing. It is used to restore spontaneous breathing and circulation of patients. Cardiac arrest refers to the sudden stop of the heart, caused by various reasons, in unexpected circumstances and time, thereby resulting in the sudden suspension of the effective cardiac pump function and effective circulation and causing serious ischemia, hypoxia and metabolic disorders of systemic histocytes. The patient may immediately lose the life without timely rescue. Cardiac arrest is different from any heart stop in the terminal stage of chronic diseases. If correct and effective resuscitation measures are taken in time, the person in distress may be saved and recovered. Since this century, China's major natural disasters have caused 130,636 people dead or missing, and direct economic losses of 6636.84 billion yuan. The treatment of critically injured patients in the emergency scene is the core and difficulty of the entire rescue mission.

The inventor believes that the above related technologies have the following defects: once cardiac arrest occurs, if no immediate and timely rescue and resuscitation are made, irreversible damage may be made to the brain of the patient and other vital organs and tissues of the person in distress after 4-6 min. Therefore, cardiopulmonary resuscitation after cardiac arrest must be carried out immediately on the scene to gain the most precious time for further rescuing and saving the lives of patients with cardiac arrest. At present, there is no cardiopulmonary resuscitation robot in China. The existing cardiopulmonary resuscitation device only has simple compression or compression ventilation function, and does not use monitoring data to form closed-loop feedback compression. The quality of cardiopulmonary resuscitation is not high, and the success rate of treatment is almost the same as that of manual compression treatment. Patients with cardiac arrest and critically injured patients at the scene of emergencies shall be treated by cardiopulmonary resuscitation, monitoring, defibrillation, ventilation and other means, so rescuers need to carry multiple equipment to the scene. However, the number of rescue equipment carried by the rescuers in the process of on-site rescue is limited. Thus, there is room for improvement in the efficient on-site rescue.

### SUMMARY

To simplify the operation complexity of medical staff in the process of cardiopulmonary resuscitation and achieve high efficiency of cardiopulmonary resuscitation rescue, the present application provides a multifunctional cardiopulmonary resuscitation machinery equipment and an operation method thereof.

The present application provides a multifunctional cardiopulmonary resuscitation machinery equipment and an operation method thereof, and adopts the following technical solution:

A multifunctional cardiopulmonary resuscitation machinery equipment comprises a first-aid kit body, and a pressing device, a ventilation device, a monitoring device, a defibrillation device and a control device which are installed on the first-aid kit body; the first-aid kit body is formed by integrated injection molding; the upper end surface of the first-aid kit body is concavely provided with a curved pillow opening on which the head of a patient is placed; the first-aid kit body is covered with a protecting pad; a belt is counter-pulled between the edges of both sides of the inner side surface of the protecting pad; the pressing device and the defibrillation device are integrated and sleeved on the belt; the ventilation device comprises an oxygen generation pump, an oxygen mask and a gas transmission pipe which are integrated inside the first-aid kit body, and an output port of the oxygen generation pump penetrates through the first-aid kit body and is connected with the oxygen mask; the monitoring device comprises two groups of patch electrical signal sensors and connected signal lines; each group of the sensors and the first-aid kit body are electrically connected through the signal lines; the control device comprises a panel installed on one side of the first-aid kit body; the panel displays physiological parameters of ECG, blood oxygen, pulse and body temperature in real time; and a battery for supplying power is integrated in the first-aid kit body.

By using the above technical solution, a portable multifunctional cardiopulmonary resuscitation machinery equipment that can be used for on-site rescue is developed in the technical solution of the present application. The integrally injection-molded first-aid kit body is used as the main body of the equipment, and the battery can supply power for each assembly. The equipment integrates chest compression, mechanical ventilation, physiological monitoring and external defibrillation effectively. The overall structure adopts a folding structure. The protecting pad can be wound on the outer side of the first-aid kit body, and becomes the form of a backpack after being folded, which is convenient for rescue personnel to go to the scene for rescue. During on-site rescue, the person in distress lies down on the protecting pad, the oxygen generation pump is started, the mouth of the person in distress is covered with the oxygen mask, and first aid is performed according to a certain first-aid sequence. At this time, each group of the sensors is pasted on the body of the person in distress and relevant parameters are set on the panel in the control device. Whole process data (compression time, frequency, depth, interval events, downtime, startup time, start time, end time, mechanical ventilation tidal volume, blowing time, blowing volume, monitoring physical data, abnormal data, defibrillation time and defibrillation energy) for patient treatment of the multifunctional cardiopulmonary resuscitation machinery equipment can be stored locally and simultaneously uploaded to a rear command system or hospital database to form an integrated life comprehensive treatment platform system. At the same time, all modules of chest compression, mechanical ventilation, physiological monitoring and external defibrillation are independent and systemically connected, and can be designed and combined flexibly according to the actual needs of on-site rescue, to facilitate rapid rescue, facilitate timely and safe rescue of the patient, reduce the mortality and disability rate, simplify the operation complexity of medical personnel during cardiopulmonary resuscitation and realize high efficiency of cardiopulmonary resuscitation rescue.

Preferably, a neck pillow sliding sleeve with a bearing bush structure is rotatably installed inside the curved pillow opening; an arc surface of one side of the neck pillow sliding sleeve away from the first-aid kit body is integrally formed with non-slip grains; an end surface of one side of the first-aid kit body away from the protecting pad is holed and fixed with a spring bolt; and an annular surface of one side of the neck pillow sliding sleeve close to the spring bolt is holed and in inserting fit with the spring bolt.

By using the above technical solution, the neck pillow sliding sleeve is designed in the position of the curved pillow opening. When the rescue personnel find debris that needs to be cleaned up in the mouth of the person in distress, the rescue personnel pull out the spring bolt, and the neck pillow sliding sleeve located inside the curved pillow opening is in a free state. The head of the person in distress falls on the neck pillow sliding sleeve. The rescue personnel rotate the neck of the person in distress. At this time, the head of the person in distress is constantly rotated. The above process improves the convenience of cleaning up foreign matter in the mouth of the person in distress, and reduces the damage to the person in distress during postural adjustment.

Preferably, a plurality of groups of sponge pads distributed in parallel along the length direction are sewn on the surface of the protecting pad, and the upper end surfaces of the sponge pads are horizontally coplanar with the lowest point of the inner peripheral surface of the neck pillow sliding sleeve.

By using the above technical solution, each group of sponge pads on the surface of the protecting pad has the function of buffering and protection, and the upper end surfaces of the sponge pads are kept horizontal with the lowest point of the inner peripheral surface of the neck pillow sliding sleeve. When the person in distress lies flat on the protecting pad, the head and the back of the person in distress are ensured to be on the basic horizontal plane. The above arrangement ensures that the airway of the person in distress after lying flat is smooth and facilitates oxygen delivery and exhaust.

Preferably, one side of the first-aid kit body is concavely provided with a notch matched with the shape of the panel, the upper position of the notch is opened and hinged and provided with a positioning block, and the end surface of one side of the positioning block away from a hinging shaft is rotatably connected with the panel.

By using the above technical solution, the positioning block is hinged and connected with the upper position of the notch, and the end surface of one side of the positioning block away from the hinging shaft is rotatably connected. The above arrangement realizes the up and down turning and rotation of the panel, so that the panel can be stored inside the notch in a daily storage state, and can be smoothly rotated to the upper end surface of the first-aid kit body during use, which is convenient for the rescue personnel to set and grasp the relevant physiological parameters of the person in distress as soon as possible.

Preferably, the protecting pad is a sealed bag structure, one side of the protecting pad is connected with an air nozzle, and the oxygen mask inflates the protecting pad through the air nozzle.

By using the above technical solution, the air nozzle is installed on the protecting pad. In the practical rescue process, some persons in distress are shocked due to drowning. At this moment, the distress location is far from the shore, so on-site rescue is needed. The protecting pad is inflated through the air nozzle. At this time, the protecting pad can float on the water under the buoyancy of the protecting pad. Thus, the persons in distress can be placed on the protecting pad, and the protecting pad plays a supporting role for the persons in distress.

Preferably, one end of the protecting pad away from the first-aid kit body is fixedly embedded with two groups of magnets, the length of the protecting pad is not less than three times the width size of the first-aid kit body, and a vertical end surface of one side of the first-aid kit body away from the protecting pad connection is fixedly embedded with iron blocks fitted and opposite to the groups of magnets.

By using the above technical solution, the magnets are fixedly embedded on the protecting pad, and the length of the protecting pad is set as above. When the equipment is in a storage state, the protecting pad is wound around the first-aid kit body, and the iron blocks of the first-aid kit body are magnetically adsorbed with the magnets on the protecting pad. The above arrangement realizes the connection between the protecting pad and the first-aid kit body. The protecting pad wrapped at the periphery of the first-aid kit body plays a role of protecting the first-aid kit body.

Preferably, the vertical end surface of one side of the first-aid kit body is connected with two groups of shoulder straps buckled with the belt respectively.

By using the above technical solution, each group of the shoulder straps is connected with the vertical end surface of one side of the first-aid kit body, and the shoulder straps are connected with the belt. With the above coordination, the person in distress on the protecting pad is not easy to fall off.

Preferably, the belt is elastic and the length of the belt is not greater than the sum of twice the length and the width of the first-aid kit body.

By using the above technical solution, when the belt is strapped on the body of the person in distress, the elastic belt can be kept fitted with the person in distress. In the storage state, the belt is turned over at this time, and the belt can wrap the protecting pad at the periphery of the first-aid kit body, thereby further improving the fitting between the protecting pad and the first-aid kit body.

Preferably, circular holes for lifting are penetrated at the corner positions of the head end and the tail end of the protecting pad.

By using the above technical solution, after the first cardiac resuscitation rescue of the person in distress at the scene is completed, the person in distress needs to be transferred to the hospital for further examination. Each group of circular holes is located at the corner positions of the head and the tail of the protecting pad. At this time, the rescue personnel can pull each group of circular holes by hands to lift the protecting pad, thereby improving the convenience of transferring the person in distress by means of the protecting pad.

Preferably, an operation method of the multifunctional cardiopulmonary resuscitation machinery equipment comprises:
a. in a daily state, winding the protecting pad on the first-aid kit body, and packing the machinery equipment into a roll; charging the battery in advance; and at this time, storing the oxygen mask, the defibrillation device and the two groups of patch electrical signal sensors at the position of the curved pillow opening;
b. carrying the equipment to the rescue scene by the rescue personnel, patting the face and shoulders of a patient, and shouting in both ears to observe whether there is a response; indicating loss of consciousness if there is no response; quickly observing whether the patient is breathing autonomously, and observing whether the thorax of the person in distress is fluctuating; simultaneously judging whether there is pulse, with judgment time of 5-10 seconds; if the patient has no consciousness, pulse and autonomous respiration, implementing cardiopulmonary resuscitation; at this time, disassembling the belt from the first-aid kit body, and paving the protecting pad on the ground; in the case of water distress, firstly connecting the oxygen mask with the air nozzle; and starting the oxygen generation pump through the panel to inflate the protecting pad;
c. moving the person in distress to the protecting pad, and then making the head and the neck pillow sliding sleeve in contact; loosening the collar and the belt to discharge objects inside the mouth of the person in distress; at this time, disconnecting the spring bolt from the opening on the neck pillow sliding sleeve; then rotating the body of the person in distress, and rotating the head to one side to facilitate the removal of foreign matter in the mouth; hermetically buckling the oxygen mask on the mouth of the person in distress, and realizing oxygen therapy and air pumping by the oxygen generation pump within a certain period;
d. integrating and sleeving the pressing device and the defibrillation device on the belt, and bundling and fixing the belt on the waist of the person in distress; stretching two groups of shoulder straps to the position of the belt and fastening with the belt; attaching two groups of patch electrical signal sensors to the heart position and the left lower abdomen of the person in distress respectively, and displaying the physiological parameters of ECG, blood oxygen, pulse and body temperature on the panel in real time;
e. taking 30 chest compressions and 2 oxygen deliveries as one cycle, and taking 5 cycles (about 2 minutes) as a group; if consciousness is not restored after 5 cycles, continuing CPR for 5 cycles until consciousness is restored or medical personnel arrive; and determining the specific time according to the specific situation of the patient;
f. after the person in distress restores consciousness, continuing to supply oxygen by the oxygen mask, loosening the belt, removing the two groups of shoulder straps, and continuing to detect the physiological parameters for 5-8 minutes; finally, disassembling the patch electrical signal sensors; lifting the person in distress from the equipment; ultimately, storing the oxygen mask and the patch electrical signal sensors towards the position of the curved pillow opening; storing the panel inside the notch by means of the positioning block; and finally rolling the protecting pad, and reversely fixing the belt with the first-aid kit body to complete the packing and storage of the machinery equipment.

By using the above technical solution, when the equipment is in the storage state, the belt wraps the first-aid kit body, which is convenient for the rescue personnel to carry and transfer the equipment. When participating in a rescue task, the rescue personnel carry the equipment to the scene. At this time, the rescue personnel remove the protecting pad from the first-aid kit body. For the rescue scene in the water body, the protecting pad needs to be inflated at first by means of the air nozzle and the oxygen mask. At this time, the protecting pad is unfolded and the person in distress lies on the protecting pad. The rescue personnel proceed according to a certain sequence of cardiopulmonary resuscitation, including autonomous awareness judgment, oral foreign body removal, clothes loosening, etc., and then begin official rescue. The defibrillation device is sleeved on the belt and two groups of shoulder straps are fixed. The two groups of sensors are attached to the body of the person in distress. At this time, the panel in the control device is started, and the physiological parameters of ECG, blood oxygen, pulse and body temperature are displayed on the panel in real time. All modules of chest compression, mechanical ventilation, physiological monitoring and external defibrillation are integrated in the above process of technical combination. Compared with the existing groups of devices which are all independent device shapes, the above equipment and the operation method thereof can significantly improve the treatment efficiency and treatment success rate of the cardiopulmonary resuscitation process.

In conclusion, the present application has at least one of the following beneficial technical effects:
1. All modules of chest compression, mechanical ventilation, physiological monitoring and external defibrillation are integrated on the equipment, and the protecting pad can be wrapped around the outer side of the first-aid kit body. The heart is effectively squeezed by using the theory of the heart pump and the chest pump in the present application to pump more blood flow. The strap compression mode has no “hammering” effect of the traditional point compression, which reduces the harm to the patient, and the strap is designed to increase the proportion of the strap around the chest to achieve a true 3D compression effect. The defibrillation device is sleeved on the belt. The automatic defibrillation technology based on the dual-phase exponential attenuation wave technology reduces the electrical current flow through the heart during defibrillation and effectively reduces the damage to myocardial tissue. A multi-feature fusion ECG rhythm detection algorithm ensures that the diagnosis accuracy is more than 95%.
2. The neck pillow sliding sleeve is slidably installed at the position of the curved pillow opening. When the rescue personnel find debris that needs to be cleaned up in the mouth of the person in distress, the rescue personnel pull out the spring bolt, and the neck pillow sliding sleeve located inside the curved pillow opening is in a free state. The head of the person in distress falls on the neck pillow sliding sleeve. The rescue personnel rotate the neck of the person in distress. At this time, the head of the person in distress is constantly rotated. The above process improves the convenience of cleaning up foreign matter in the mouth of the person in distress, and reduces the damage to the person in distress during postural adjustment.
3. The air nozzle is installed on the pad. In the practical rescue process, some persons in distress are shocked due to drowning. At this moment, the distress location is far from the shore, so on-site rescue is needed. The protecting pad is inflated through the air nozzle. At this time, the protecting pad can float on the water under the buoyancy of the protecting pad. Thus, the persons in distress can be placed on the protecting pad, and the protecting pad plays a supporting role for the persons in distress.

### DESCRIPTION OF DRAWINGS

FIG. 1 is an integral structural schematic diagram of an embodiment of the present application;

FIG. 2 is an enlarged view of part A in FIG. 1;

FIG. 3 is an integral structural schematic diagram of an embodiment of the present application in another view;

FIG. 4 is an enlarged view of part B in FIG. 3;

FIG. 5 is an integral structural schematic diagram in a storage state in the present application.

### REFERENCE SIGNS

1. first-aid kit body; 11. curved pillow opening; 12. neck pillow sliding sleeve; 121. non-slip grains; 13. spring bolt; 14. notch; 141. positioning block; 15 iron sheet; 2. pressing device; 3. ventilation device; 31. oxygen generation pump; 32. oxygen mask; 33. gas transmission pipe; 4. monitoring device; 41. sensor; 42. signal line; 5. defibrillation device; 6. control device; 61. panel; 7. protecting pad; 71. sponge pad; 72. air nozzle; 73. magnet; 74. circular hole; 8. belt; 81. shoulder strap; 9. battery; 10. person in distress; 101. head.

### DETAILED DESCRIPTION

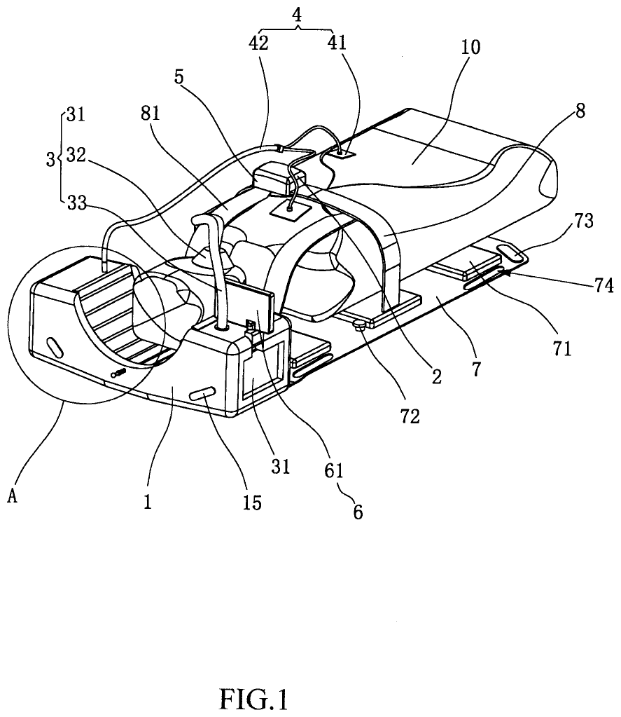

The present application is further described below in detail in combination with FIGS. 1-5.

### Embodiment 1

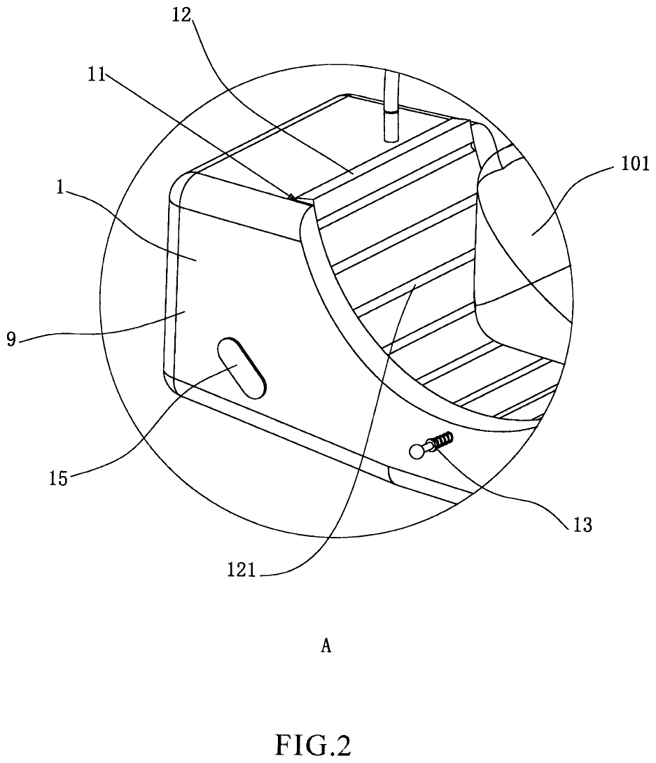

The embodiment of the present application discloses a multifunctional cardiopulmonary resuscitation machinery equipment. By referring to FIG. 1 and FIG. 2, the multifunctional cardiopulmonary resuscitation machinery equipment comprises a first-aid kit body 1, and a pressing device 2, a ventilation device 3, a monitoring device 4, a defibrillation device 5 and a control device 6 which are installed on the first-aid kit body 1. The first-aid kit body 1 is formed by integrated injection molding. The size of the first-aid kit body 1 is preferably length×width×height=600 mm×250 mm×250 mm. The upper end surface of the first-aid kit body 1 is concavely provided with a curved pillow opening 11 on which the head 101 of a patient is placed. The caliber of the curved pillow opening 11 is not less than 400 mm. A neck pillow sliding sleeve 12 with a bearing bush structure is rotatably installed inside the curved pillow opening 11, and an arc surface of one side of the neck pillow sliding sleeve 12 away from the first-aid kit body 1 is integrally formed with non-slip grains 121. The first-aid kit body 1 is covered with a protecting pad 7, and the protecting pad 7 is connected with the lower edge of one side of the first-aid kit body 1 in the length direction. Circular holes 74 for lifting are penetrated at the corner positions of the head end and the tail end of the protecting pad 7. After the first cardiac resuscitation rescue of the person 10 in distress at the scene is completed, the person 10 in distress needs to be transferred to the hospital for further examination. Each group of circular holes 74 is located at the corner positions of the head and the tail of the protecting pad 7. At this time, the rescue personnel can pull each group of circular holes 74 by hands to lift the protecting pad 7, thereby improving the convenience of transferring the person 10 in distress by means of the protecting pad 7. The width of the protecting pad 7 is adapted with the length of the first-aid kit body 1. A belt 8 is counter-pulled between the edges of both sides of the inner side surface of the protecting pad 7; and the pressing device 2 and the defibrillation device 5 are integrated and sleeved on the belt 8.

The vertical end surface of one side of the first-aid kit body 1 is connected with two groups of shoulder straps 81 buckled with the belt 8 respectively. Each group of the shoulder straps 81 is connected with the vertical end surface of one side of the first-aid kit body 1, and the shoulder straps 81 are connected with the belt 8. With the above coordination, the person 10 in distress on the protecting pad 7 is not easy to fall off. The belt 8 is elastic and the length of the belt 8 is not greater than the sum of twice the length and the width of the first-aid kit body 1. The belt 8 can be kept fitted with the person 10 in distress. In the storage state, the belt 8 is turned over at this time, and the belt 8 can wrap the protecting pad 7 at the periphery of the first-aid kit body 1, thereby further improving the fitting between the protecting pad 7 and the first-aid kit body 1.

An end surface of one side of the first-aid kit body 1 away from the protecting pad 7 is holed and fixed with a spring bolt 13; and an annular surface of one side of the neck pillow sliding sleeve 12 close to the spring bolt 13 is holed and in inserting fit with the spring bolt 13. When the rescue personnel find debris that needs to be cleaned up in the mouth of the person 10 in distress, the rescue personnel pull out the spring bolt 13, and the neck pillow sliding sleeve 12 located inside the curved pillow opening 11 is in a free state. The head 101 of the person 10 in distress falls on the neck pillow sliding sleeve 12. The rescue personnel rotate the neck of the person 10 in distress. At this time, the head 101 of the person 10 in distress is constantly rotated. The above process improves the convenience of cleaning up foreign matter in the mouth of the person 10 in distress, and reduces the damage to the person 10 in distress during postural adjustment.

The ventilation device 3 comprises an oxygen generation pump 31, an oxygen mask 32 and a gas transmission pipe 33 which are integrated inside the first-aid kit body 1, and an output port of the oxygen generation pump 31 penetrates through the first-aid kit body 1 and is connected with the oxygen mask 32 through the gas transmission pipe 33; the monitoring device 4 comprises two groups of patch electrical signal sensors 41; each group of the sensors 41 and the first-aid kit body 1 are electrically connected through the signal lines 42; the control device 6 comprises a panel 61 installed on one side of the first-aid kit body 1; the panel 61 displays physiological parameters of ECG, blood oxygen, pulse and body temperature in real time; and a battery 9 for supplying power is integrated in the first-aid kit body 1 (the battery 9 in the figure is integrated in the first-aid kit body 1, and a line points to the first-aid kit body 1 for simple illustration, which belongs to the prior art in this field and is not repeated).

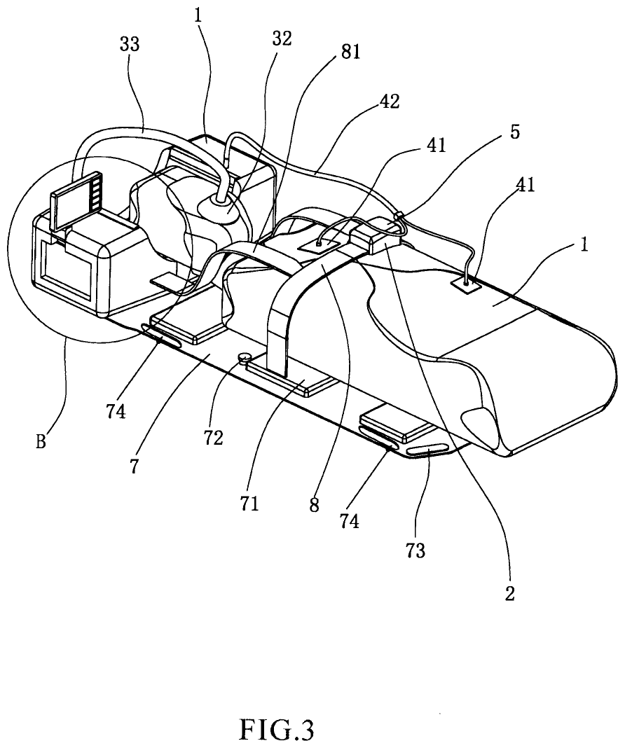

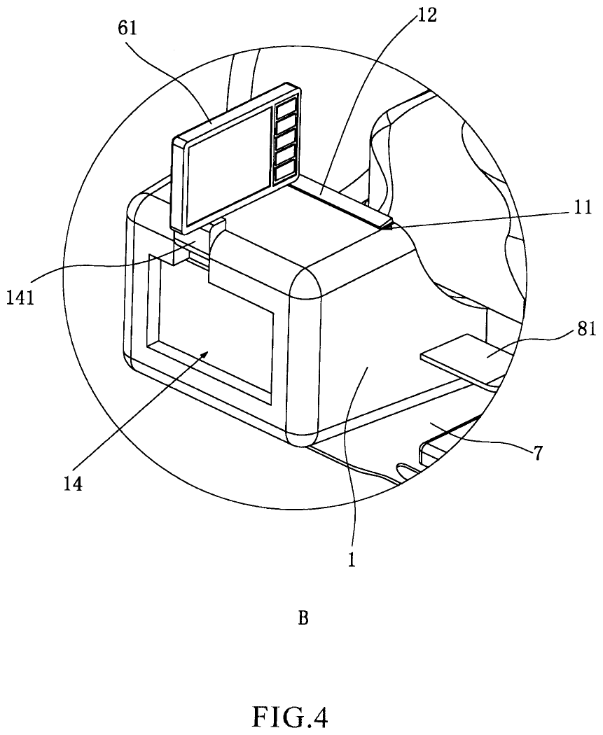

By referring to FIG. 3 and FIG. 4, a plurality of groups of sponge pads 71 distributed in parallel along the length direction are sewn on the surface of the protecting pad 7, each group of sponge pads 71 penetrates through the protecting pad 7, and the upper end surfaces of the sponge pads 71 are horizontally coplanar with the lowest point of the inner peripheral surface of the neck pillow sliding sleeve 12. Each group of sponge pads 71 has the function of buffering and protection, and the upper end surfaces of the sponge pads 71 are kept horizontal with the lowest point of the inner peripheral surface of the neck pillow sliding sleeve 12. When the person 10 in distress lies flat on the protecting pad 7, the head 101 and the back of the person 10 in distress are ensured to be on the basic horizontal plane. The above arrangement ensures that the airway of the person 10 in distress after lying flat is smooth and facilitates oxygen delivery and exhaust. The protecting pad 7 is a sealed bag structure, one side of the protecting pad 7 is connected with an air nozzle 72, and the oxygen mask 32 inflates the protecting pad 7 through the air nozzle 72. In the practical rescue process, some persons 10 in distress are shocked due to drowning. At this moment, the distress location is far from the shore, so on-site rescue is needed. The protecting pad 7 is inflated through the air nozzle 72. At this time, the protecting pad 7 can float on the water under the buoyancy of the protecting pad 7. Thus, the persons 10 in distress can be placed on the protecting pad 7, and the protecting pad 7 plays a supporting role for the persons 10 in distress.

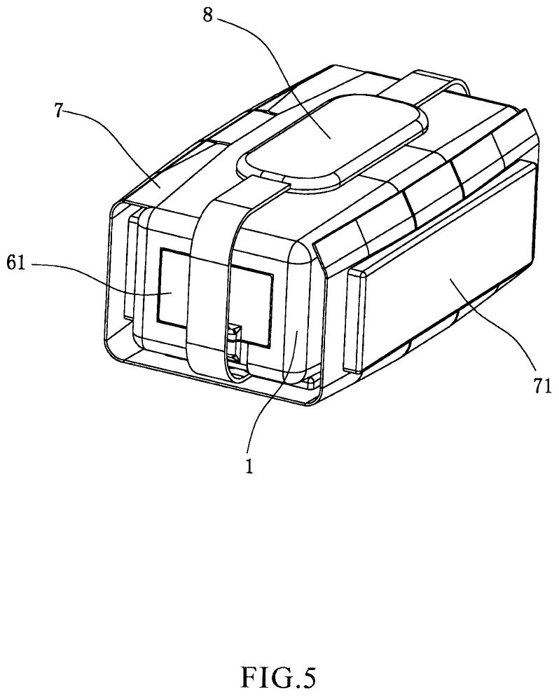

One side of the first-aid kit body 1 is concavely provided with a notch 14 matched with the shape of the panel 61, the upper position of the notch 14 is opened and hinged and provided with a positioning block 141, and the end surface of one side of the positioning block 141 away from a hinging shaft is rotatably connected. The above arrangement realizes the up and down turning and rotation of the panel 61, so that the panel 61 can be stored inside the notch 14 in a daily storage state, and can be smoothly rotated to the upper end surface of the first-aid kit body 1 during use, which is convenient for the rescue personnel to set and grasp the relevant physiological parameters of the person 10 in distress as soon as possible. One end of the protecting pad 7 away from the first-aid kit body 1 is fixedly embedded with two groups of magnets 73, the length of the protecting pad 7 is not less than three times the width size of the first-aid kit body 1, and a vertical end surface of one side of the first-aid kit body 1 away from the connection of the protecting pad 7 is fixedly embedded with iron blocks 15 fitted and opposite to the groups of magnets 73. By referring to FIG. 5, when the equipment is in a storage state, the protecting pad 7 is wound around the first-aid kit body 1, and the iron blocks of the first-aid kit body 1 are magnetically adsorbed with the magnets 73 on the protecting pad 7. The above arrangement realizes the connection between the protecting pad 7 and the first-aid kit body 1. The protecting pad 7 wrapped at the periphery of the first-aid kit body 1 plays a role of protecting the first-aid kit body 1.

### Embodiment 2

An operation method of the multifunctional cardiopulmonary resuscitation machinery equipment comprises:
a. in a daily state, winding the protecting pad 7 on the first-aid kit body 1, and packing the machinery equipment into a roll; charging the battery 9 in advance; and at this time, storing the oxygen mask 32, the defibrillation device 5 and the two groups of patch electrical signal sensors 41 at the position of the curved pillow opening 11;
b. carrying the equipment to the rescue scene by the rescue personnel, patting the face and shoulders of a patient, and shouting in both ears to observe whether there is a response; indicating loss of consciousness if there is no response; quickly observing whether the patient is breathing autonomously, and observing whether the thorax of the person 10 in distress is fluctuating; simultaneously judging whether there is pulse, with judgment time of 5-10 seconds; if the patient has no consciousness, pulse and autonomous respiration, implementing cardiopulmonary resuscitation; at this time, disassembling the belt 8 from the first-aid kit body 1, and paving the protecting pad 7 on the ground; in the case of water distress, firstly connecting the oxygen mask 32 with the air nozzle 72; and starting the oxygen generation pump 31 through the panel 61 to inflate the protecting pad 7;
c. moving the person 10 in distress to the protecting pad 7, and then making the head 101 and the neck pillow sliding sleeve 12 in contact; loosening the collar and the belt 8 to discharge objects inside the mouth of the person 10 in distress; at this time, disconnecting the spring bolt 13 from the opening on the neck pillow sliding sleeve 12; then rotating the body of the person 10 in distress, and rotating the head 101 to one side to facilitate the removal of foreign matter in the mouth; hermetically buckling the oxygen mask 32 on the mouth of the person 10 in distress, and realizing oxygen therapy and air pumping by the oxygen generation pump 31 within a certain period;
d. integrating and sleeving the pressing device 2 and the defibrillation device 5 on the belt 8, and bundling and fixing the belt 8 on the waist of the person 10 in distress; stretching two groups of shoulder straps 81 to the position of the belt 8 and fastening with the belt 8; attaching two groups of patch electrical signal sensors 41 to the heart position and the left lower abdomen of the person 10 in distress respectively, and displaying the physiological parameters of ECG, blood oxygen, pulse and body temperature on the panel 61 in real time;
e. taking 30 chest compressions and 2 oxygen deliveries as one cycle, and taking 5 cycles (about 2 minutes) as a group; if consciousness is not restored after 5 cycles, continuing CPR for 5 cycles until consciousness is restored or medical personnel arrive; and determining the specific time according to the specific situation of the patient;
f. after the person 10 in distress restores consciousness, continuing to supply oxygen by the oxygen mask 32, loosening the belt 8, removing the two groups of shoulder straps 81, and continuing to detect the physiological parameters for 5-8 minutes; finally, disassembling the patch electrical signal sensors 41; lifting the person 10 in distress from the equipment; ultimately, storing the oxygen mask 32 and the patch electrical signal sensors 41 towards the position of the curved pillow opening 11; storing the panel 61 inside the notch 14 by means of the positioning block 141; and finally rolling the protecting pad 7, and reversely fixing the belt 8 with the first-aid kit body 1 to complete the packing and storage of the machinery equipment.

A portable multifunctional cardiopulmonary resuscitation machinery equipment that can be used for on-site rescue is developed in the technical solution of the present application. The integrally injection-molded first-aid kit body 1 is used as the main body of the equipment, and the battery 9 can supply power for each assembly. The equipment integrates chest compression, mechanical ventilation, physiological monitoring and external defibrillation effectively. The overall structure adopts a folding structure. The protecting pad 7 can be wound on the outer side of the first-aid kit body 1, and becomes the form of a backpack after being folded, which is convenient for rescue personnel to go to the scene for rescue. During on-site rescue, the person 10 in distress lies down on the protecting pad 7, the oxygen generation pump 31 is started, the mouth of the person 10 in distress is covered with the oxygen mask 32, and first aid is performed according to a certain first-aid sequence. At this time, each group of the sensors 41 is pasted on the body of the person 10 in distress and relevant parameters are set on the panel 61 in the control device 6. Whole process data (compression time, frequency, depth, interval events, downtime, startup time, start time, end time, mechanical ventilation tidal volume, blowing time, blowing volume, monitoring physical data, abnormal data, defibrillation time and defibrillation energy) for patient treatment of the multifunctional cardiopulmonary resuscitation machinery equipment can be stored locally and simultaneously uploaded to a rear command system or hospital database to form an integrated life comprehensive treatment platform system. At the same time, all modules of chest compression, mechanical ventilation, physiological monitoring and external defibrillation are independent and systemically connected, and can be designed and combined flexibly according to the actual needs of on-site rescue, to facilitate rapid rescue, facilitate timely and safe rescue of the patient, reduce the mortality and disability rate, simplify the operation complexity of medical personnel during cardiopulmonary resuscitation and realize high efficiency of cardiopulmonary resuscitation rescue.

The above illustrates the preferred embodiments of the present application and is not intended to limit the protection scope of the present application. Therefore, any equivalent change made in accordance with the structure, shape and principle of the present application shall be covered within the protection scope of the present application.

What is claimed is:

1. A multifunctional cardiopulmonary resuscitation machinery equipment, comprising a first-aid kit body (1), and a pressing device (2), a ventilation device (3), a monitoring device (4), a defibrillation device (5) and a control device (6) which are installed on the first-aid kit body (1), wherein the first-aid kit body (1) is formed by integrated injection molding; the upper end surface of the first-aid kit body (1) is concavely provided with a curved pillow opening (11) on which the head of a patient is placed; the first-aid kit body (1) is covered with a protecting pad (7); a belt (8) is counter-pulled between the edges of both sides of the inner side surface of the protecting pad (7); the pressing device (2) and the defibrillation device (5) are integrated and sleeved on the belt (8); the ventilation device (3) comprises an oxygen generation pump (31), an oxygen mask (32) and a gas transmission pipe (33) which are integrated inside the first-aid kit body (1), and an output port of the oxygen generation pump (31) penetrates through the first-aid kit body (1) and is connected with the oxygen mask (32); the monitoring device (4) comprises two groups of patch electrical signal sensors (41) and connected signal lines (42); each group of the sensors (41) and the first-aid kit body (1) are electrically connected through the signal lines (42); the control device (6) comprises a panel (61) installed on one side of the first-aid kit body (1); the panel (61) displays physiological parameters of ECG, blood oxygen, pulse and body temperature in real time; and a battery (9) for supplying power is integrated in the first-aid kit body (1).

2. The multifunctional cardiopulmonary resuscitation machinery equipment according to claim 1, wherein a neck pillow sliding sleeve (12) with a bearing bush structure is rotatably installed inside the curved pillow opening (11); an arc surface of one side of the neck pillow sliding sleeve (12) away from the first-aid kit body (1) is integrally formed with non-slip grains (121); an end surface of one side of the first-aid kit body (1) away from the protecting pad (7) is holed and fixed with a spring bolt (13); and an annular surface of one side of the neck pillow sliding sleeve (12) close to the spring bolt (13) is holed and in inserting fit with the spring bolt (13).

3. The multifunctional cardiopulmonary resuscitation machinery equipment according to claim 2, wherein a plurality of groups of sponge pads (71) distributed in parallel along the length direction are sewn on the surface of the protecting pad (7), and the upper end surfaces of the sponge pads (71) are horizontally coplanar with the lowest point of the inner peripheral surface of the neck pillow sliding sleeve (12).

4. The multifunctional cardiopulmonary resuscitation machinery equipment according to claim 3, wherein one side of the first-aid kit body (1) is concavely provided with a notch (14) matched with the shape of the panel (61), the upper position of the notch (14) is opened and hinged and provided with a positioning block (141), and the end surface of one side of the positioning block (141) away from a hinging shaft is rotatably connected with the panel (61).

5. The multifunctional cardiopulmonary resuscitation machinery equipment according to claim 4, wherein the protecting pad (7) is a sealed bag structure, one side of the protecting pad (7) is connected with an air nozzle (72), and the oxygen mask (32) inflates the protecting pad (7) through the air nozzle (72).

6. The multifunctional cardiopulmonary resuscitation machinery equipment according to claim 5, wherein one end of the protecting pad (7) away from the first-aid kit body (1) is fixedly embedded with two groups of magnets (73), the length of the protecting pad (7) is not less than three times the width size of the first-aid kit body (1), and a vertical end surface of one side of the first-aid kit body (1) away from the connection of the protecting pad (7) is fixedly embedded with iron blocks (15) fitted and opposite to the groups of magnets (73).

7. The multifunctional cardiopulmonary resuscitation machinery equipment according to claim 1, wherein the vertical end surface of one side of the first-aid kit body (1) is connected with two groups of shoulder straps (81) buckled with the belt (8) respectively.

8. The multifunctional cardiopulmonary resuscitation machinery equipment according to claim 7, wherein the belt (8) is elastic and the length of the belt (8) is not greater than the sum of twice the length and the width of the first-aid kit body (1).

9. The multifunctional cardiopulmonary resuscitation machinery equipment according to claim 6, wherein circular holes (74) for lifting are penetrated at the corner positions of the head end and the tail end of the protecting pad (7).

10. An operation method of the multifunctional cardiopulmonary resuscitation machinery equipment, comprising:
a. in a daily state, winding the protecting pad (7) on the first-aid kit body (1), and packing the machinery equipment into a roll; charging the battery (9) in advance; and at this time, storing the oxygen mask (32), the defibrillation device (5) and the two groups of patch electrical signal sensors (41) at the position of the curved pillow opening (11);
b. carrying the equipment to the rescue scene by the rescue personnel, patting the face and shoulders of a patient, and shouting in both ears to observe whether there is a response; indicating loss of consciousness if there is no response; quickly observing whether the patient is breathing autonomously, and observing whether the thorax of the person in distress is fluctuating; simultaneously judging whether there is pulse, with judgment time of 5-10 seconds; if the patient has no consciousness, pulse and autonomous respiration, implementing cardiopulmonary resuscitation; at this time, disassembling the belt (8) from the first-aid kit body (1), and paving the protecting pad (7) on the ground; in the case of water distress, firstly connecting the oxygen mask (32) with the air nozzle (72); and starting the oxygen generation pump (31) through the panel (61) to inflate the protecting pad (7);
c. moving the person in distress to the protecting pad (7), and then making the head and the neck pillow sliding sleeve (12) in contact; loosening the collar and the belt (8) to discharge objects inside the mouth of the person in distress; at this time, disconnecting the spring bolt (13) from the opening on the neck pillow sliding sleeve (12); then rotating the body of the person in distress, and rotating the head to one side to facilitate the removal of foreign matter in the mouth; hermetically buckling the oxygen mask (32) on the mouth of the person in distress, and realizing oxygen therapy and air pumping by the oxygen generation pump (31) within a certain period;
d. integrating and sleeving the pressing device (2) and the defibrillation device (5) on the belt (8), and bundling and fixing the belt (8) on the waist of the person in distress; stretching two groups of shoulder straps (81) to the position of the belt (8) and fastening with the belt (8); attaching two groups of patch electrical signal sensors (41) to the heart position and the left lower abdomen of the person in distress respectively, and displaying the physiological parameters of ECG, blood oxygen, pulse and body temperature on the panel (61) in real time;
e. taking 30 chest compressions and 2 oxygen deliveries as one cycle, and taking 5 cycles (about 2 minutes) as a group; if consciousness is not restored after 5 cycles, continuing CPR for 5 cycles until consciousness is restored or medical personnel arrive; and determining the specific time according to the specific situation of the patient;
f. after the person in distress restores consciousness, continuing to supply oxygen by the oxygen mask (32), loosening the belt (8), removing the two groups of shoulder straps (81), and continuing to detect the physiological parameters for 5-8 minutes; finally, disassembling the patch electrical signal sensors (41); lifting the person in distress from the equipment; ultimately, storing the oxygen mask (32) and the patch electrical signal sensors (41) towards the position of the curved pillow opening (11); storing the panel (61) inside the notch (14) by means of the positioning block (141); and finally rolling the protecting pad (7), and reversely fixing the belt (8) with the first-aid kit body (1) to complete the packing and storage of the machinery equipment.
